(19) United States
(12) Patent Application Publication
(10) Pub. No.: US 2004/0242565 A1
(43) Pub. Date: Dec. 2, 2004

(54) Medicinal composition for prevention of or treatment for cerebrovascular disorder and cardiopathy

(76) Inventors: Yoshinori Toshima, Shizuoka (JP); Asako Hitomi, Shizuoka (JP); Shin-ichi Satoh, Shizuoka (JP); Ichiro Ikegaki, Shizuoka (JP)

Correspondence Address: BIRCH STEWART KOLASCH & BIRCH, PO BOX 747, FALLS CHURCH, VA, 22040-0747, US

(21) Appl. No.: 10/488,699

(22) PCT Filed: Jul. 30, 2002
(86) PCT No.: PCT/JP02/07712

(30) Foreign Application Priority Data
Sep. 11, 2001 (JP) 2001-274846

(51) Int. Cl. A61K 31/551

(52) U.S. Cl. 514/218

(57) ABSTRACT

A pharmaceutical composition comprising at least one of components (a) and at least one of components (b) shown in below:

(a) a compound represented by the general formula (I)

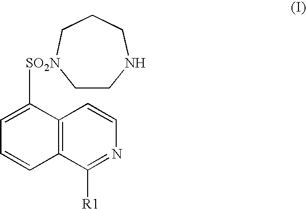

(wherein R1 represents a hydrogen atom or a hydroxyl group) or an acid addition salt or hydrate thereof; and

(b) an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, an immunosuppressant, or a pharmaceutically acceptable salt (except the components shown in (a)). It is useful as a preventive or remedy for cerebrovascular disorders and cardiac diseases.

### TECHNICAL FIELD

The present invention relates to a pharmaceutical composition that is effective against prevention or treatment of diseases such as a cerebrovascular disorder and a cardiac disease.

### RELATED ART

For prevention or treatment of diseases such as a cerebrovascular disorder and a cardiac disease, there have been used drugs such as an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, and an immunosuppressant.

On the other hand, it has been already known that a compound represented by the general formula (I):

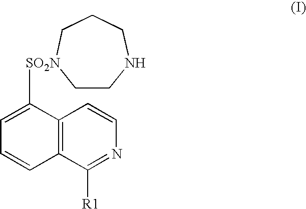

(wherein R1 represents a hydrogen atom or a hydroxyl group) has an inhibitory activity against a kinase such as a Rho kinase, a myosin light chain kinase, or a protein kinase C; shows a vascular smooth muscle relaxation effect, a blood flow increasing effect, an antihypertensive effect, a cerebral protective effect, a cardiac protective effect, or the like; and is an effective substance in a vasodilator agent (particularly as a treating agent for angina), a cerebral protecting agent, a cardiac protecting agent, or the like (for example, JP 61-227581 A, JP 02-256617 A, JP 06-056668 A, JP 07-080854 B, EP 0187371, WO 98/06433, Br. J. Pharmacol., 98, 1091 (1989), J. Pharmacol. Exp. Ther., 259, 738 (1991), Circulation, 96, 4357 (1997), Cardiovasc. Res., 43, 1029 (1999)).

In the aforementioned documents, the single use of the compound represented by the general formula (I) is disclosed. However, there have been no report regarding concomitant use or a plan of concomitant use of the compound with an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, or an immunosuppressant until the pharmaceutical composition of the present invention is disclosed.

### DISCLOSURE OF THE INVENTION

An object of the present invention is to provide a pharmaceutical composition that has a marked effect than that obtained when a preventive drug or a treatment drug is administered singly for prevention or treatment of a cerebrovascular disorder, a cardiac disease, and the like.

In view of the above-mentioned circumstances, the inventors of the present invention have found that the compound represented by the general formula (I) exerts particularly significant effect that was not obtained when administering the compound singly for, e.g., drug effect, safeness, stability, dosage, dosage form, usage, or the like by using the compound in combination with an ameliorant of cerebral circulation, avasodilator, an cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, or an immunosuppressant. Thus, the inventors have accomplished the present invention based on the knowledge.

That is, the present invention relates to the followings:

(1) A pharmaceutical composition including at least one of components (a) and at least one of components (b) shown in below.

(a) A compound represented by the following general formula (I):

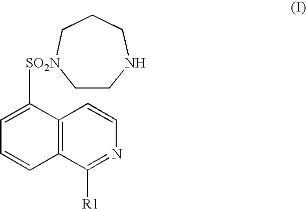

(wherein R1 represents a hydrogen atom or a hydroxyl group)

(b) An ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, and an immunosuppressant (except the components shown in (a)).

Note that, in the present invention, the components (a) include, in addition to the compound represented by the general formula (I), hydrates thereof (for example, 1/2 hydrates, 1 hydrates, and 3 hydrates) and acid addition salts thereof.

Moreover, the drug of components (b), which is used together with the component (a) represented by the general formula (I) according to the present invention, includes pharmaceutically acceptable salts thereof.

(2) The pharmaceutical composition according to item (1), in which the vasodilator shown in (b) is a calcium channel blocking drug and the calcium channel blocking drug is nimodipine.

(3) The pharmaceutical composition according to item (1), in which the thrombolytic drug shown in (b) is a tissue plasminogen activator and the tissue plasminogen activator is alteplase.

(4) The pharmaceutical composition according to item (1), in which the antiplatelet drug shown in (b) is clopidogrel or aspirin.

(5) The pharmaceutical composition according to item (1), in which the antianginal drug shown in (b) is a calcium channel blocking drug and the calcium channel blocking drug is amlodipine or nifedipine.

(6) The pharmaceutical composition according to item (1), in which the antianginal drug shown in (b) is a nitrate drug and the nitrate drug is isosorbide.

(7) The pharmaceutical composition according to item (1), in which the antianginal drug shown in (b) is a β-adrenaline receptor antagonist and the β-adrenaline receptor antagonist is atenolol.

(8) The pharmaceutical composition according to item (1), which is provided for prevention or treatment of a cerebrovascular disorder.

(9) The pharmaceutical composition according to item (8), in which the cerebrovascular accident is selected from the group consisting of: cerebral infarction, cerebral thrombosis, cerebral embolism, cerebral hemorrhage, cerebral vasospasm, subarachnoid hemorrhage, transient ischemic attack, cerebral arteriosclerosis, head trauma, and cerebral edema; and psychiatric symptom, neurologic symptom, disorder in activities of daily livingdisability, amnesia, and dementia, which are based on those diseases.

(10) The pharmaceutical composition according to item (8), in which the cerebrovascular disorder is selected from the group consisting of: cerebral infarction and cerebral vasospasm; and neurologic symptom and disorder in activities of daily living disability, which are based on those diseases.

(11) The pharmaceutical composition according to item (8), in which the cerebrovascular disorder is selected from the group consisting of: cerebral vasospasm developed after subarachnoid hemorrhage; and neurologic symptom and disorder in activities of daily living disability, which are associated with those diseases.

(12) The pharmaceutical composition according to item (2), which is provided for prevention or treatment of a cerebrovascular disorder.

(13) The pharmaceutical composition according to item (12), in which the cerebrovascular disorder is selected from the group consisting of: cerebral infarction, cerebral thrombosis, celebral embolism, transient ischemic attack, cerebral arteriosclerosis, head trauma, and cerebral edema; and psychiatric symptom, neurologic symptom, disorder in activities of daily living disability, amnesia, and dementia, which are based on those diseases.

(14) The pharmaceutical composition according to item (12), in which the cerebrovascular disorder is selected from the group consisting of: cerebral infarction and cerebral vasospasm; and a neurologic symptom and disorder in activities of daily living disability, which are based on those diseases.

(15) The pharmaceutical composition according to item (12), in which the cerebrovascular disorder is selected from the group consisting of: cerebral vasospasm developed after subarachnoid hemorrhage; and neurologic symptom and disorder in activities of daily living disability, which are associated with those diseases.

(16) The pharmaceutical composition according to item (3), which is provided for prevention or treatment of a cerebrovascular disorder.

(17) The pharmaceutical composition according to item (16) in which the cerebrovascular disorder is selected from the group consisting of: cerebral infarction, cerebral thrombosis, celebral embolism, and transient ischemic attack; and neurologic symptom and disorder in activities of daily living disability, which are based on those diseases.

(18) The pharmaceutical composition according to item (4), which is provided for prevention or treatment of a cerebrovascular disorder.

(19) The pharmaceutical composition according to item (18), in which the cerebrovascular disorder is selected from the group consisting of: cerebral infarction, cerebral thrombosis, celebral embolism, cerebral hemorrhage, cerebral vasospasm, subarachnoid hemorrhage, transient ischemic attack, cerebral arteriosclerosis, head trauma, and cerebral edema; and psychiatric symptom, neurologic symptom, disorder in activities of daily living disability, amnesia, and dementia, which are based on those diseases.

(20) The pharmaceutical composition according to item (1), which is provided for prevention or treatment of a cardiac disease.

(21) The pharmaceutical composition according to item (20), in which the cardiac disease is selected from the group consisting of myocardial ischemia, angina pectoris, myocardial infarction, complication of myocardial infarction, reperfusion injury in treatment of myocardial infarction, and cardiac failure.

(22) The pharmaceutical composition according to item (20), in which the cardiac disease is angina pectoris.

(23) The pharmaceutical composition according to item (5), which is provided for prevention or treatment of a cardiac disease.

(24) The pharmaceutical composition according to item (23), in which the cardiac disease is selected from the group consisting of myocardial ischemia, angina pectoris, myocardial infarction, complication of myocardial infarction, reperfusion injury in treatment of myocardial infarction, and cardiac failure.

(25) The pharmaceutical composition according to item (23), in which the cardiac disease is angina pectoris.

(26) The pharmaceutical composition according to item (6), which is provided for prevention or treatment of a cardiac disease.

(27) The pharmaceutical composition according to item (26), in which the cardiac disease is selected from the group consisting of myocardial ischemia, angina pectoris, myocardial infarction, complication of myocardial infarction, reperfusion injury in treatment of myocardial infarction, and cardiac failure.

(28) The pharmaceutical composition according to item (26), in which the cardiac disease is angina pectoris.

(29) The pharmaceutical composition according to item (4), which is provided for prevention or treatment of a cardiac disease.

(30) The pharmaceutical composition according to item (29), in which the cardiac disease is selected from the group consisting of myocardial ischemia, angina pectoris, myocardial infarction, complication of myocardial infarction, reperfusion injury in treatment of myocardial infarction, and cardiac failure.

(31) The pharmaceutical composition according to item (29), in which the cardiac disease is angina pectoris.

(32) The pharmaceutical composition according to item (7), which is provided for prevention or treatment of a cardiac disease.

(33) The pharmaceutical composition according to item (32), in which the cardiac disease is selected from the group consisting of myocardial ischemia, angina pectoris, myocardial infarction, complication of myocardial infarction, reperfusion injury in treatment of myocardial infarction, and cardiac failure.

(34) The pharmaceutical composition according to item (32), in which the cardiac disease is angina pectoris.

(35) A use of a certain amount of the compound shown in (a) and a certain amount of at least one treatment agent of the drugs shown in (b) or a pharmaceutically acceptable salt thereof for prevention or treatment of a cerebrovascular disorder and a cardiac disease.

(36) A method of treating a cerebrovascular disorder or a cardiac disease using a certain amount of the compound shown in (a) and a certain amount of at least one treatment agent of the components shown in (b) or a pharmaceutically acceptable salt thereof.

(37) A document which describes that a certain amount of the compound shown in (a) and a certain amount of at least one treatment agent of the drugs shown in (b) or a pharmaceutically acceptable salt thereof can or should be used for prevention or treatment of a cerebrovascular disorder and a cardiac disease; and a package including the document.

The compound of the present invention represented by the general formula (I) can be synthesized in accordance with a well known in the method described in, for example, Chem. Pharam. Bull., 40, (3) 770-773 (1992); JP 61-152658 A; or the like. In addition, an acid addition salt thereof is preferably a pharmaceutically acceptable nontoxic salt. Examples of the salt include: salts of inorganic acids such as hydrochloric acid, hydrobromic acid, phosphoric acid, and sulfuric acid; and salts of organic acids such as acetic acid, citric acid, tartaric acid, lactic acid, succinic acid, fumaric acid, maleic acid, and methanesulfonic acid.

Examples of the ameliorant of cerebral circulation to be used together with the compound of the present invention shown in (a) include ozagrel, argatroban, nicergoline, concentrated glycerin/fructose, meclofenoxate, nizofenone, dihydroergotoxine, ibudilast, ifenprodil. Examples of the vasodilator include bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin. Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of the brain metabolic stimulants include amantadine, tiapride, and γ-aminobutyric acid. Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. Examples of the thrombolytic drug include urokinase, tissue-type plasminogen activators (such as alteplase, tisokinase, nateplase, pamiteplase, monteplase, and rateplase), and nasaruplase. Examples of the amelirant of psychiatric symptom include treatment agents for schizophrenia (such asphenothiazine drugs, butyrophenone drugs, benzamide drugs, and risperidone), antianxiety agents (such as thienodiazepine drugs, benzodiazepine drugs, and tandospirone), antidepressants (fluvoxamine, milnacipran, iminobenzyl derivatives, dibenzocycloheptadiene derivatives, maprotiline, mianserin, and setiptiline), and antidementia drugs (such as donepezil). Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), a-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine. Examples of the antianginal drug include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calciumchannel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline) trimetazidine, dipyridamole, etafenone, dilazep, trapidil, nicorandil, enoxaparin, and aspirin. Examples of the diuretic include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, andpotassiumcanrenoate), osmotic diuretics (such as isosorbide, D-mannitol, and glycerin), nonthiazide diuretics (such as meticrane, tripamide, chlorthalidone, and mefruside), and acetazolamide. Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as amrinone, olprinone, and milrinone), denopamine, ubidecarenone, pimobendan, levosimendan, aminoethylsulfonic acid, vesnarinone, carperitide, and colforsin daropate. Examples of the antiarrhythmic drug include ajmaline, pirmenol, procainamide, cibenzoline, disopyramide, quinidine, aprindine, mexiletine, lidocaine, phenyloin, pilsicainide, propafenone, flecainide, atenolol, acebutolol, sotalol, propranolol, metoprolol, pindolol, amiodarone, nifekalant, diltiazem, bepridil, and verapamil. Examples of the antihyperlipidemic drug include atorvastatin, simvastatin, pravastatin sodium, fluvastatin sodium, clinofibrate, clofibrate, simfibrate, fenofibrate, bezafibrate, colestimide, and colestyramine. Examples of the immunosuppressant include azathioprine, mizoribine, cyclosporine, tacrolimus, gusperimus, and methotrexate.

In addition, examples of the drugs of the present invention shown in (b) to be used together with the compound shown in (a) or pharmaceutically acceptable salts thereof include carperitide, eplerenone, vatanidipine, lemildipine, clevidipine, zofenopril, olmesartan, KRH-594, omapatrilat, fasidotril, ecadotril, sampatrilat, MDL-100240, Z-13752A, xamoterol, molsidomine, ivabradine, cariporide, etomoxir, HMR-1098, eniporide, BIII-890, SL-65.1708, triflusal, tinzaparin, pamicogrel, tirofiban, eptifibatide, abciximab, YM-33, BGC-728, CI-1031, NAPc2, SB-249417, nesiritide, urodilatin, BG-9928, DTI-0017, monosialoganglioside GM-1, tirilazad, nicaraven, EGb-761, ebselen, NXY-059, EPC-K1, sitaxsentan, tezosentan, ambrisentan, zonampanel, enrasentan, darusentan, J-104132, BMS-207940, BSF-302146, TBC-3711, ABT-546, Ro-61-1790, aliskiren, vepalimomab, VPA-985, SR-121463, toborinone, MCC-135, MCI-154, treprostinol, repinotan, SUN-N4057, dexanabinol, UK-315716, irampanel, DY-9760e, pexelizumab, UK-279276, and NS-7.

The pharmaceutical composition of the present invention includes not only a mixture in which at least one of the above-mentioned components (a) and at least one of the above-mentioned components (b) have been mixed previously, but also a nonmixed combination such as a form of a kit or a pharmaceutical package.

On preparing the pharmaceutical composition of the present invention as a formulation having a form suitable for administration, there may be mixed: the compound represented by the general formula (I) (wherein R1 represents a hydrogen atom or a hydroxyl group) provided as an active ingredient or an acid addition salt or hydrate thereof; at least one treatment agent of the drugs shown in (b) or a pharmaceutically acceptable salt thereof; and a pharmaceutically acceptable carrier which is known as a supplementary component. Examples of the carrier include: gelatin; saccharides such as lactose and glucose; starches such as wheat, rice, and corn starch; fatty acids such as stearic acid; fatty acid salts such as calcium stearate and magnesium stearate; talc; vegetable oils; alcohols such as stearic alcohol and benzyl alcohol; gum; and polyalkylene glycol.

Moreover, general examples of a liquid carrier include: water; physiological saline; a solution containing dextrose or a similar saccharide; and glycols such as ethylene glycol, propylene glycol, polyethylene glycol, and polypropylene glycol. When the pharmaceutical composition of the present invention is prepared as a capsule, typically, gelatin is preferably used.

The pharmaceutical composition of the present invention, which is composed of: the carrier provided as a supplementary component; the compound represented by the general formula (I) (wherein R1 represents a hydrogen atom or a hydroxyl group) provided as an active ingredient or an acid addition salt or hydrate thereof; and at least one treatment agent of the drugs shown in (b) or a pharmaceutically acceptable salt thereof, includes an active ingredient in an amount of, typically 0.01 wt % or more and 80 wt % or less, preferably 60 wt % or less, for example.

In addition, the present invention relates to a method of treating a cerebrovascular disorder or a cardiac disease using a certain amount of the compound shown in (a) above and a certain amount of at least one treatment agent of the drugs shown in (b) or a pharmaceutically acceptable salt thereof. Those may be simultaneously administered as a mixture and may be separately administered successively as a different pharmaceutical form. In the case of the repetitive administration, the time interval between administrations is preferably 48 hours or less. Moreover, the compound shown in (a) and the drug shown in (b) may have different administration routes from each other.

That is, when one has an oral administration, and the other may have a parenteral administration.

Examples of the administration method include oral administration and parenteral administration. Examples of a dosage form suitable for the oral administration include a tablet, a capsule, a powder, a granule, a liquid agent, and an elixir.

Examples of a dosage form suitable for the parenteral administration include a liquid agent.

When the pharmaceutical composition is parenterally administered by intramuscular injection, intravenous injection, or subcutaneous injection, the composition can be administered in the form of a sterile solution that is added with another solute such as sodium chloride or glucose. When the pharmaceutical composition is administered by injection, the composition is preferably dissolved in sterilized water, a lidocaine hydrochloride solution (for intramuscular injection), physiological saline, glucose, a solution for intravenous injection, or an electrolyte solution (for intravenous injection).

When the composition is dissolved in such a manner, the solution may be regulated so as to contain an active ingredient in an amount of, typically 0.01 wt % or more and 20 wt % or less, preferably 0.1 wt % or more and 10 wt % or less. In the case of a liquid agent for oral administration, preferable examples include a suspension or syrup that contains an active ingredient in an amount of 0.01 to 20 wt %. Examples of a carrier to be used in such a case include an aqueous diluents such as perfume, syrup, or pharmaceutical micell.

The dosage of the pharmaceutical composition of the present invention depends on age of a patient to be administered, health condition, body weight, degree of symptom, type of treatment if any simultaneous treatment is performed, frequency of treatment, property of desired effect, administration route, or administration plan. Typically, effective dosage of the compound of the present invention represented by the general formula (I) (wherein R1 represents a hydrogen atom or a hydroxyl group) or an acid addition salt or hydrate thereof is 0.01 to 20 mg/kg·day in the case of parenteral administration, and 0.02 to 40 mg/kg·day in the case of oral administration. Effective dosage of the drug shown in (b) or a pharmaceutically acceptable salt thereof is typically 0.0001 to 100 mg/kg·day. In addition, effective dosage in the case where the vasodilator shown in (b) is a calcium channel blocking drug and the calcium channel blocking drug is nimodipine is 0.1 to 3 mg/kg·day in the case of parenteral administration, and 0.3 to 30 mg/kg·day in the case of oral administration.

The present invention also provides a kit, package, or document for convenient and effective use of the pharmaceutical composition of the present invention. That is, the present invention relates to a medical kit for prevention or treatment of a cerebrovascular disorder and a cardiac disease, which is characterized by including a first container containing a certain amount of the compound shown in (a) and a second container containing a certain amount of at least one treatment agent of the drugs shown in (b) or a pharmaceutically acceptable salt thereof. By using such a kit, the present invention facilitates a use of the pharmaceutical composition of the present invention, and also facilitates accurate administration of a suitable active ingredient to a patient with accurate dosage by a physician.

Moreover, other means that facilitate to understand and use the pharmaceutical composition of the present invention include a briefing document and a package. That is, a document which describes the use of a certain amount of the compound shown in (a) together with a certain amount of at least one treatment agent of the drugs shown in (b) or a pharmaceutically acceptable salt thereof for prevention or treatment of a cerebrovascular disorder and a cardiac disease; and a package which includes the document. The document and the package which includes the document facilitate to understand and use the pharmaceutical composition of the present invention, and also facilitates accurate administration of a suitable active ingredient to a patient with accurate dosage by a physician.

### BEST MODE FOR CARRYING OUT THE INVENTION

The present invention will next be described more specifically by way of examples, formulation example, and kit example. However, the present invention is not limited thereto.

### EXAMPLE 1

### Effect on Dog Delayed Cerebral Vasospasm Model

An adult mongrel dog was anesthetized with pentobarbital and 5 ml of autologous blood was injected into cisterna magna (on the first day). On the third day, 5 ml of autologous blood was injected into cisterna magna under thiamyral anesthesia. Before the injection of autologous blood on the first day and on the seventh day, angiographin was injected into vertebral artery, followed by photographing blood vessel of basilar artery. The blood vessel photograph on the seventh day confirmed occurrence of delayed cerebral vasospasm on the photograph. Then, a hydrochloride of the compound represented by the general formula (I) (wherein R1 represents a hydrogen atom) (0.3 mg/kg) and nimodipine (0.1 mg/kg or 1 mg/kg) were separately administered into vein over 30 minutes continuously (separate administration group). Alternatively, a hydrochloride of the compound represented by the general formula (I) (wherein R1 represents ahydrogen atom) (0.3 mg/kg) and nimodipine (0.1 mg/kg) were concomitantly administered into vein over 30 minutes continuously (concomitant administration group). As a control, physiological saline was administered into vein over 30 minutes continuously. After the administration of each drug, the blood vessel was photographed to measure the diameter of basilar artery. Table 1 shows the percentage (%) of the blood vessel diameter after the administration of each drug based on the blood vessel diameter before the injection of autologous blood (on the first day).

TABLE 1

| | Basilar artery diameter |
|---|---|
| | (diameter before blood |
| Compound | injection: 100%) |
| Physiological saline | 59.9% |
| Hydrochloride of general formula (I) | 60.5% |
| compound (wherein R1 represents a hydrogen | |
| atom) 0.3 mg/kg | |
| Nimodipine 0.1 mg/kg | 55.7% |
| Nimodipine 1 mg/kg | 58.0% |
| Hydrochloride of general formula (I) | 68.8% |
| compound (wherein R1 represents a hydrogen | |
| atom) 0.3 mg/kg + Nimodipine 0.1 mg/kg | |

The diameter of basilar artery measured before the drug administration (on the seventh day) decreased to about 60% compared with that measured before the autologous blood injection (on the first day), and occurrence of delayed cerebral vasospasm was confirmed. When the hydrochloride of the compound represented by the general formula (I) (wherein R1 represents a hydrogen atom) (0.3 mg/kg) and nimodipine (0.1 mg/kg or 1 mg/kg) were separately administered, the vasospasm was not improved. When the hydrochloride of a compound represented by the general formula (I) (wherein R1 represents a hydrogen atom) (0.3 mg/kg) and nimodipine (0.1 mg/kg or 1 mg/kg) were concomitantly administered, an vasospasm improving effect was observed.

Also, an vasospasm improving effect was observed in the case of the concomitant administration of a certain amount of the hydrochloride and nimodipine that had not caused vasospasm improving effect when administering those separately.

Those results confirmed that the concomitant administration of the hydrochloride of the compound represented by the general formula (I) (wherein R1 represents a hydrogen atom) and nimodipine caused a synergistic effect. Also, those results indicated that the concomitant administration of the compound represented by the general formula (I) (wherein R1 represents a hydrogen atom) and nimodipine was effective for improvement and prevention of cerebral vasospasm.

### EXAMPLE 2

### Inhibitory Effect of Cerebral Infarction in Rat Cerebral Infarction Model

A rat brain microthromboembolism model described in Stroke, 31, 2245-2255 (2000) was used as a cerebral infarction model. Physiological saline, a hydrochloride of a compound represented by the general formula (I) (wherein R1 represents a hydrogen atom) (1 mg/kg), or sodium ozagrel provided as an ameliorant of cerebral circulation (10 mg/kg) was separately administered intraperitoneally to a rat provided as a cerebral infarction model (separate administration group) Alternatively, a hydrochloride of a compound represented by the general formula (I) (wherein R1 represents a hydrogen atom) (1 mg/kg) and sodium ozagrel (10 mg/kg) were administered intraperitoneally (concomitant administration group). After the model was prepared, each drug was administered once a day until the fourth day. On the fifth day, the brain was extracted and the size of a cerebral infarction was histopathologically measured. In the case of the separate administration of the hydrochloride of the compound represented by the general formula (I) (wherein R1 represents a hydrogen atom) (1 mg/kg) and sodium ozagrel (10 mg/kg), the cerebral infarction was not inhibited. On the other hand, in the case of the concomitant administration of the hydrochloride of the compound represented by the general formula (I) (wherein R1 represents a hydrogen atom) (1 mg/kg) and sodium ozagrel (10 mg/kg), the cerebral infarction was inhibited. Also, the inhibitory effect of cerebral infarction was confirmed in the case of the concomitant administration of a certain amount of the hydrochloride and sodium ozagrel that had not caused the inhibitory effect of cerebral infarction when administering those separately. It was confirmed that the concomitant administration of the hydrochloride of the compound represented by the general formula (I) (wherein R1 represents a hydrogen atom) and sodium ozagrel caused a synergistic effect. It was indicated that the concomitant administration of the compound represented by the general formula (I) (wherein R1 represents a hydrogen atom) and sodium ozagrel was effective for improvement and prevention of cerebral infarction.

### EXAMPLE 3

### Effect on Neuronal Death Model by Transient Occlusion of Both Common Carotid Arteries of Mongolian Gerbil

Both common carotid arteries of a Mongolian gerbil were occluded for 5 minutes to cause transient brain ischemic condition. Immediately after restarting blood flow, a hydrochloride of the compound represented by the general formula (I) (wherein R1 represents a hydrogen atom) (0.3 mg/kg) or nimodipine (3 mg/kg or 10 mg/kg) was separately administered intraperitoneally (separate administration group).

Alternatively, a hydrochloride of the compound represented by the general formula (I) (wherein R1 represents a hydrogen atom) (0.3 mg/kg) and nimodipine (0.3 mg/kg) were administered intraperitoneally (concomitant administration group).

On the seventh day, the number of pyramidal cells in a hippocampus CA1 region was counted. The brain ischemia decreased the number of pyramidal cells to about 10%. The separate administration of the hydrochloride of the compound represented by the general formula (I) (wherein R1 represents a hydrogen atom) (0.3 mg/kg) and nimodipine (3 mg/kg) did not inhibit neuronal death. The concomitant administration of the hydrochloride of the compound represented by the general formula (I) (wherein R1 represents a hydrogen atom) (0.3 mg/kg) and nimodipine (3 mg/kg) inhibited neuronal death. Also, the brain inhibitory effect of neuronal death was confirmed in the case of the concomitant administration of a certain amount of the hydrochloride and nimodipine that had not caused the brain inhibitory effect of neuronal death when administering those separately.

Those results confirmed that the concomitant administration of the hydrochloride of the compound represented by the general formula (I) (wherein R1 represents a hydrogen atom) and nimodipine caused a synergistic effect. In addition, those results indicated that the concomitant administration of the compound represented by the general formula (I) (wherein R1 represents a hydrogen atom) and nimodipine was effective for improvement and prevention of cerebral infarction.

### EXAMPLE 4

### Effect on Vasopressin Induced Rat Angina Pectoris Model

A rat was orally administered with physiological saline, a hydrochloride of the compound represented by the general formula (I) (wherein, R1 is a hydrogen atom) (3 mg/kg), nifedipine provided as a calcium channel blocking drug (3 mg/kg), propranolol provided as a β-adrenaline receptor blocking drug (100 mg/kg), or isosorbide nitrate provided as a nitrate drug (30 mg/kg) separately (separate administration group). Alternatively, a rat was orally administered with one compound of a hydrochloride of the compound represented by the general formula (I) (wherein, R1 is a hydrogen atom) (3 mg/kg), nifedipine (3 mg/kg), propranolol (100 mg/kg), and isosorbide nitrate (30 mg/kg) (concomitant administration group). Half an hour later, vasopressin (0.5 U/kg) was intravenously administered. The ST segment depression was used as an index showing the degree of a myocardial ischemia. On the seventh day, the ST segment in an electrocardiogram was measured for comparison with a ST segment before the vasopressin administration. It was found that the vasopressin administration caused the ST segment depression. In addition, occurrence of a myocardial ischemia was observed. The ST segment depression was not improved by the separate administration of the hydrochloride of the compound represented by the general formula (I) (wherein, R1 is a hydrogen atom), nifedipine, propranolol, and isosorbide nitrate. The ST segment depression was improved by the concomitant administration of: the hydrochloride of the compound represented by the general formula (I) (wherein, R1 is a hydrogen atom) and nifedipine; the hydrochloride of the compound represented by the general formula (I) (wherein, R1 is a hydrogen atom) and propranolol; or the hydrochloride of the compound represented by the general formula (I) (wherein, R1 is a hydrogen atom) and isosorbide nitrate. The improving effect of myocardial ischemia was confirmed in the case of the concomitant administration of a certain amount of the hydrochloride, nifedipine, propranolol, and isosorbide nitrate that had not caused the improving effect of myocardial ischemia when administering those separately. Those results confirmed that the concomitant administration of any of the hydrochloride of the compound represented by the general formula (I) (wherein R1 represents a hydrogen atom), nifedipine, propranolol, and isosorbide nitrate caused a synergistic effect. In addition, those results indicated that the concomitant administration of any of the hydrochloride of the compound represented by the general formula (I) (wherein R1 represents a hydrogen atom), nifedipine, propranolol, and isosorbide nitrate was effective for improvement and prevention of angina pectoris.

### FORMULATION EXAMPLE

A pharmaceutical composition, which includes (a), a compound represented by the general formula (I):

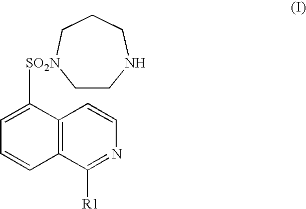

(wherein R1 represents a hydrogen atom or a hydroxyl group) or an acid addition salt or hydrate thereof and which includes at least one of (b) an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, and an immunosuppressant can be produced in accordance with, for example, the formulation shown below.

1. Sterile Injectable

A preferable amount of at least one of the drugs shown in (b) was added to the components shown in Table 2 below and those compounds were dissolved in distilled water for injection. Subsequently, the solution was regulated to have a required final weight by addition of distilled water for injection and an ampule containing 2 ml of the solution was sealed, followed by heat sterilization.

TABLE 2

| | | Active ingredient |
|---|---|---|
| | Component | content |
| 10 mg formulation | Hydrochloride of general | 10 mg |
| | formula (I) compound | |
| | (wherein R1 represents a | |
| | hydrogen atom) | |
| | Sodium chloride | 16 mg |
| | Distilled water | Proper amount |
| | | Total amount was |
| | | regulated to 2 ml |
| 30 mg formulation | Hydrochloride of general | 30 mg |
| | formula (I) compound | |
| | (wherein R1 represents a | |
| | hydrogen atom) | |
| | Sodium chloride | 16 mg |
| | Distilled water | Proper amount |
| | | Total amount was |
| | | regulated to 2 ml |
| 60 mg formulation | Hydrochloride of general | 60 mg |
| | formula (I) compound | |
| | (wherein R1 represents a | |
| | hydrogen atom) | |
| | Sodium chloride | 16 mg |
| | Distilled water | Proper amount |
| | | Total amount was |
| | | regulated to 2 ml |
| 10 mg formulation | Hydrochloride of general | 10 mg |
| | formula (I) compound | |
| | (wherein R1 represents a | |
| | hydroxyl group) | |
| | Sodium chloride | 16 mg |
| | Distilled water | Proper amount |
| | | Total amount was |
| | | regulated to 2 ml |
| 30 mg formulation | Hydrochloride of general | 30 mg |
| | formula (I) compound | |
| | (wherein R1 represents a | |
| | hydroxyl group) | |
| | Sodium chloride | 16 mg |
| | Distilled water | Proper amount |
| | | Total amount was |
| | | regulated to 2 ml |
| 60 mg formulation | Hydrochloride of general | 60 mg |
| | formula (I) compound | |
| | (wherein, R1 | |
| | represents a hydroxyl | |
| | group) | |
| | Sodium chloride | 16 mg |
| | Distilled water | Proper amount |
| | | Total amount was |
| | | regulated to 2 ml |

2. Tablet

A preferable amount of at least one of the drugs shown in (b) was added to the components shown in Table 3 below to prepare a tablet in accordance with a general method.

TABLE 3

| | Component | Amount |
|---|---|---|
| 10 mg formulation | Hydrochloride of general formula | 10.0 mg |
| | (I) compound (wherein R1 | |
| | represents a hydrogen atom) | |
| | Crystalline cellulose | 25.0 mg |
| | Lactose | 108.5 mg |
| | Magnesium stearate | 1.5 mg |
| | Carboxymethylcellulose calcium | 5.0 mg |
| | Total | 150.0 mg |
| 20 mg formulation | Hydrochloride of general formula | 20.0 mg |
| | (I) compound (wherein R1 | |
| | represents a hydrogen atom) | |
| | Crystalline cellulose | 25.0 mg |
| | Lactose | 98.5 mg |
| | Magnesium stearate | 1.5 mg |
| | Carboxymethylcellulose calcium | 5.0 mg |
| | Total | 150.0 mg |
| 10 mg formulation | Hydrochloride of general formula | 10.0 mg |
| | (I) compound (wherein R1 | |
| | represents a hydroxyl group) | |
| | Crystalline cellulose | 25.0 mg |
| | Lactose | 108.5 mg |
| | Magnesium stearate | 1.5 mg |
| | Carboxymethylcellulose calcium | 5.0 mg |
| | Total | 150.0 mg |
| 20 mg formulation | Hydrochloride of general formula | 20.0 mg |
| | (I) compound (wherein R1 | |
| | represents a hydroxyl group) | |
| | Crystalline cellulose | 25.0 mg |
| | Lactose | 98.5 mg |
| | Magnesium stearate | 1.5 mg |
| | Carboxymethylcellulose calcium | 5.0 mg |
| | Total | 150.0 mg |

### KIT EXAMPLE

A kit of pharmaceutical composition, which includes (a), a compound represented by the general formula (I):

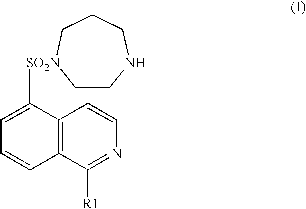

(wherein R1 represents a hydrogen atom or a hydroxyl group) or an acid addition salt or hydrate thereof and which includes at least one of (b) an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, and an immunosuppressant can be provided in a form shown below, for example.

At least one of pharmaceutical dosage form units shown in Table 4 and at least one of pharmaceutical dosage form units shown in Table 5 were separately packed in a divided bottle or a divided foil packet, and the bottle or the packet was stored in a package container.

TABLE 4

| | |
|---|---|
| | Active |
| | ingredient |
| Component | content |

1. Ampule agent

| | | |
|---|---|---|
| 10 mg formulation | Hydrochloride of general | 10 mg |
| | formula (I) compound (wherein | |
| | R1 represents a hydrogen atom) | |
| 30 mg formulation | Hydrochloride of general | 30 mg |
| | formula (I) compound (wherein | |
| | R1 represents a hydrogen atom) | |
| 60 mg formulation | Hydrochloride of general | 60 mg |
| | formula (I) compound (wherein | |
| | R1 represents a hydrogen atom) | |
| 10 mg formulation | Hydrochloride of general | 10 mg |
| | formula (I) compound (wherein | |
| | R1 represents a hydroxyl group) | |
| 30 mg formulation | Hydrochloride of general | 30 mg |
| | formula (I) compound (wherein | |
| | R1 represents a hydroxyl group) | |
| 60 mg formulation | Hydrochloride of general | 60 mg |
| | formula (I) compound (wherein | |
| | R1 represents a hydroxyl group) | |

2. Tablet

| | | |
|---|---|---|
| 10 mg formulation | Hydrochloride of general | 10.0 mg |
| | formula (I) compound (wherein | |
| | R1 represents a hydrogen atom) | |
| 20 mg formulation | Hydrochloride of general | 20.0 mg |
| | formula (I) compound (wherein | |
| | R1 represents a hydrogen atom) | |
| 10 mg formulation | Hydrochloride of general | 10.0 mg |
| | formula (I) compound (wherein | |
| | R1 represents a hydroxyl group) | |
| 20 mg formulation | Hydrochloride of general | 20.0 mg |
| | formula (I) compound (wherein | |
| | R1 represents a hydroxyl group) | |

TABLE 5

| | |
|---|---|
| | Active |
| | ingredient |
| Component | content |

1. Ampule agent

| | | |
|---|---|---|
| 10 mg formulation | Nimodipine | 10 mg |

2. Tablet

| | | |
|---|---|---|
| 30 mg formulation | Nimodipine | 30 mg |
| 60 mg formulation | Nimodipine | 60 mg |
| 100 mg formulation | Nimodipine | 100 mg |

3. capsule

| | | |
|---|---|---|
| 30 mg formulation | Nimodipine | 30 mg |

### INDUSTRIAL APPLICABILITY

The present invention can provide a pharmaceutical composition that is useful as a preventive or treatment is useful as a preventive or treatment agent for a cerebrovascular disorder and a cardiac disease.

1. A pharmaceutical composition comprising at least one of components (a) and at least one of components (b) shown in below:
(a) a compound represented by the general formula (I) 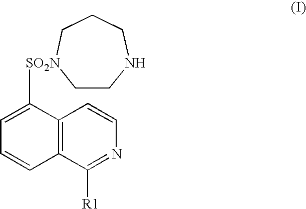
(wherein R1 represents a hydrogen atom or a hydroxyl group) or an acid addition salt or hydrate thereof; and
(b) an ameliorant of cerebral circulation, a vasodilator, a cerebral protecting drug, an brain metabolic stimulants, an anticoagulant, an antiplatelet drug, a thrombolytic drug, an amelirant of psychiatric symptom, a antihypertensive drug, an antianginal drug, a diuretic, a cardiotonic, an antiarrhythmic drug, an antihyperlipidemic drug, an immunosuppressant, or a pharmaceutically acceptable salt (except the components shown in (a)).

2. A pharmaceutical composition according to claim 1, which is provided for prevention or treatment of a cerebrovascular disorder.

3. A pharmaceutical composition according to claim 2, wherein the cerebrovascular disorder is selected from the group consisting of: cerebral infarction, cerebral thrombosis, cerebral embolism, cerebral hemorrhage, cerebral vasospasm, subarachnoid hemorrhage, transient ischemic attack, cerebral arteriosclerosis, head trauma, and cerebral edema; and psychiatric symptom, neurologic symptom, disorder in activities of daily living disability, amnesia, and dementia, which are based on those diseases.

4. A pharmaceutical composition according to claim 3, wherein sodium ozagrel is used as an ameliorant of cerebral circulation shown in claim 1 (b).

5. A pharmaceutical composition according to claim 3, wherein nimodipine is used as a vasodilator shown in claim 1 (b).

6. A pharmaceutical composition according to claim 1, which is provided for prevention or treatment of a cardiac disease.

7. A pharmaceutical composition according to claim 6, wherein the cardiac disease is selected from the group consisting of myocardial ischemia, angina pectoris, myocardial infarction, complication of myocardial infarction, reperfusion injury in myocardial infarct treatment, and cardiac failure.

8. A pharmaceutical composition according to claim 7, wherein nifedipine, propranolol, or isosorbide nitrate is used as an antianginal drug shown in claim 1 (b).
